(19) United States
(12) Patent Application Publication
(10) Pub. No.: US 2021/0212759 A1
(43) Pub. Date: Jul. 15, 2021

(54) MEDICAL DEVICE

(71) Applicant: TERUMO KABUSHIKI KAISHA, Tokyo (JP)

(72) Inventors: Tuo ZHOU, Yokohama-shi (JP); Itaru OKUBO, Naka-gun Ninomiya-cho (JP)

(73) Assignee: TERUMO KABUSHIKI KAISHA, Tokyo (JP)

(21) Appl. No.: 17/213,525

(22) Filed: Mar. 26, 2021

Related U.S. Application Data
(63) Continuation of application No. PCT/JP2019/038318, filed on Sep. 27, 2019.

(30) Foreign Application Priority Data
Sep. 27, 2018 (JP) 2018-181976

(51) Int. Cl. A61B 18/14

(52) U.S. Cl. CPC A61B 18/1492; A61B 2018/00267

(57) ABSTRACT

A medical device which can be inserted into a tapered biological lumen and can effectively ablate a wide range. The medical device includes an elongated shaft, a plurality of electrically independent electrodes disposed in a distal portion of the elongated shaft, the plurality of electrically independent electrodes extending along an axial direction of the shaft portion and deformable in a radial direction of the elongated shaft, and a plurality of electrically independent conductors including an embedded portion embedded in the elongated shaft, the plurality of electrically independent conductors and configured to allow a current to flow to the plurality of electrodes. At least one of the plurality of electrically independent conductors has a protruding portion protruding from a distal surface of the shaft portion, and connected to one of the plurality of electrically independent electrodes.

### CROSS-REFERENCES TO RELATED APPLICATIONS

This application is a continuation of International Application No. PCT/JP2019/038318 filed on Sep. 27, 2019, which claims priority to Japanese Patent Application No. 2018-181976 filed on Sep. 27, 2018, the entire content of both of which is incorporated herein by reference.

### FIELD OF THE DISCLOSURE

The present disclosure generally relates to a medical device inserted into a living body to perform ablation treatment on a biological tissue.

### BACKGROUND DISCUSSION

Medical devices that perform an irreversible electroporation (IRE) treatment are known. The irreversible electroporation has attracted attention since the treatment is non-thermal and can help suppress damage to surrounding blood vessels or nerves. For example, a medical device is known in which a cancer that is less likely to be removed by surgery is treated by using the irreversible electroporation.

For atrial fibrillation caused by abnormal excitement appearing in a myocardial sleeve of a pulmonary vein wall, pulmonary vein isolation may be performed to destroy myocardial cells by ablating a joint portion between a pulmonary vein and a left atrium. In the pulmonary vein isolation, high frequency waves are generated from a distal end of an ablation catheter to cauterize and necrotize a myocardium in a dot shape. The ablation catheter is moved to cauterize an inflow portion of the pulmonary vein in a circumferential shape, and isolates the pulmonary vein.

For example, U.S. Pat. No. 9,227,036 discloses a device in which a ring-shaped electrode is provided on an outer peripheral surface of an elongated tubular body. A conductor for supplying a current to the electrode is spirally disposed inside the tubular body.

It is desirable that a device inserted into a biological lumen has a reduced diameter so that the device can be inserted into a relatively narrow biological lumen. However, according to the device disclosed in U.S. Pat. No. 9,227,036 described above, an electrode is located outside a tubular body with respect to a conductor in a radial direction. Therefore, it can be difficult to reduce a diameter of the device inserted into a living body.

### SUMMARY

A medical device is disclosed which can be inserted into a narrow biological lumen and can effectively ablate a relatively wide range.

A medical device is disclosed, which includes an elongated shaft portion, a plurality of electrically independent electrode portions disposed on a distal side of the shaft portion, extending along a length direction of the shaft portion, and deformable in a radial direction of the shaft portion, and a plurality of electrically independent conductors including an embedded portion embedded in the shaft portion, and allowing a current to flow to the electrode portions. At least one of the conductors has a protruding portion protruding from a distal surface of the shaft portion, and connected to the electrode portion.

In accordance with an aspect, a medical device is disclosed comprising: an elongated shaft; a plurality of electrically independent electrodes disposed on a distal side of the elongated shaft, the plurality of electrically independent electrodes extending along an axial direction of the elongated shaft and deformable in a radial direction of the elongated shaft; a plurality of electrically independent conductors, each of the plurality of electrically independent conductors including an embedded portion embedded in the elongated shaft, and the plurality of electrically independent conductors configured to allow a current to flow to the plurality of electrically independent electrodes; and wherein at least one of the plurality of electrically independent conductors has a protruding portion protruding from a distal surface of the elongated shaft portion, and wherein the at least one of the plurality of electrical independent conductors is connected to one of the plurality of electrically independent electrodes.

In accordance with another aspect, a medical device is disclosed comprising: an elongated shaft that includes an outer tubular body and an inner tubular body, the inner tubular body extending inside of the outer tubular body and along the outer tubular body; a plurality of electrically independent electrodes disposed distal of the elongated shaft portion, the plurality of electrically independent electrodes extending along an axial direction of the shaft portion and deformable in a radial direction of the elongated shaft; a plurality of electrically independent conductors including a portion extending along the shaft portion and disposed between an outer surface of the inner tube body and an inner surface of the outer tube body, the portion of the plurality of electrically independent conductors extending along the elongated shaft being configured to allow a current to flow to at least one of the plurality of electrically independent electrodes; the inner tubular body including an extending portion that extends distal of the distal end of the outer tubular body; and wherein at least one of the plurality of electrically independent conductors includes a protruding portion protruding from the distal end of the outer tubular body, and the protruding portion is connected to at least one of the plurality of electrically independent electrodes on the extending portion of the inner tubular body.

In accordance with a further aspect, a medical device is disclosed comprising: an elongated shaft that includes a first tubular body; a plurality of electrodes disposed distal of the shaft portion, the plurality of electrodes extending along a length direction of the elongated shaft and deformable in a radial direction of the shaft portion; a plurality of conductors extending on an outer surface of the first tubular body along the shaft portion and include connection sections at distal end portions of the conductors, the connection sections electrically connected to proximal end sections of the plurality of electrodes; an annular support body disposed on an outer peripheral surface of the first tubular body; an insulation tube formed of an insulation material and disposed on the annular support body; the support body including a plurality of housing portions aligned in a circumferential direction of the support body, the housing portions housing the proximal end sections of the plurality of electrodes; and wherein the proximal end sections of the plurality of electrodes and the connection sections of the plurality of conductors are disposed on the first tubular body and covered together with the support body by the insulation tube.

According to the medical device configured as described above, the electrode portion does not protrude outward in the radial direction, and can have a reduced diameter. Therefore, the medical device can be inserted into a relatively narrow biological lumen, and a relatively wide range can be effectively ablated by the electrode portion curved in the radial direction.

### BRIEF DESCRIPTION OF THE DRAWINGS

FIG. 1 is a front view illustrating a medical device according to a first exemplary embodiment.

FIG. 2A is a sectional view of the medical device taken along line IIA-IIA in FIG. 1.

FIG. 2B is a sectional view of the medical device taken along line IIB-IIB in FIG. 1.

FIG. 3 is a sectional view illustrating a distal portion of the medical device.

FIG. 4 is a circumferential development view of a shaft portion which illustrates a conductor through an outer tube in a transparent view.

FIG. 5 is a sectional view illustrating the distal portion of the medical device in a state where an electrode portion is expanded.

FIG. 6 is front view illustrating a medical device according to a second exemplary embodiment.

FIG. 7 is a sectional view taken along line VII-VII in FIG. 6.

FIG. 8 is a sectional view illustrating a shaft portion.

FIG. 9 is a sectional view illustrating a distal portion of a medical device according to a third exemplary embodiment.

FIG. 10 is a sectional view taken along line X-X in FIG. 9.

### DETAILED DESCRIPTION

Set forth below with reference to the accompanying drawings is a detailed description of embodiments of a medical device inserted into a living body to perform ablation treatment on a biological tissue representing examples of the inventive medical device. Note that since embodiments described below are preferred specific examples of the present disclosure, although various technically preferable limitations are given, the scope of the present disclosure is not limited to the embodiments unless otherwise specified in the following descriptions. Dimensions in the drawings are exaggerated and different from actual dimensions for convenience of description, in some cases. In addition, in the description herein and the drawings, the same reference numerals will be assigned to configuration elements having substantially the same functional configuration, and thus, repeated description will be omitted. In the description herein, a side where a device is inserted into a lumen will be referred to as a “distal side”, and an operating hand-side will be referred to as a “proximal side”.

### First Embodiment

A medical device 10 according to a first exemplary embodiment is percutaneously inserted into a biological lumen, comes into contact with a biological tissue of a target site, and applies an electric signal to perform irreversible electroporation. A target of the medical device 10 of the present exemplary embodiment is an electroporation treatment performed over an entire periphery of an entrance portion of a pulmonary vein in pulmonary vein isolation. However, the medical device 10 is also applicable to other treatments.

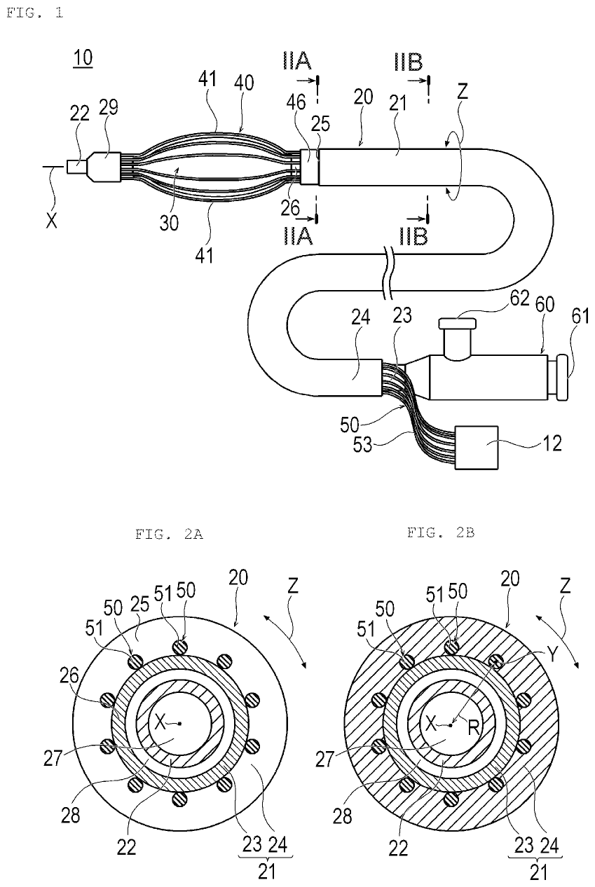

As illustrated in FIGS. 1 to 3, the medical device 10 has an elongated shaft portion 20, a balloon 30 which is an expansion body provided in a distal portion of the shaft portion 20, and a hub 60 provided in a proximal portion of the shaft portion 20. Furthermore, the medical device 10 has a plurality of electrode portions 40 provided around the balloon 30 and a conductor 50 that transmits a current to the electrode portions 40.

The shaft portion 20 has a tubular outer tube 21 and a tubular inner tube 22 disposed inside a first tubular body 23 of the tubular outer tube 21. The outer tube 21 and the inner tube 22 are disposed coaxially with each other. The outer tube 21 and the inner tube 22 are relatively movable in an axial direction. The outer tube 21 includes the first tubular body 23 and a second tubular body 24 which covers an outer peripheral surface of the first tubular body 23 and is fixed to the first tubular body 23. The conductor 50 is disposed to be interposed between the first tubular body 23 and the second tubular body 24. The first tubular body 23 and the second tubular body 24 are disposed coaxially with each other. The first tubular body 23 has a step portion 26 extending to a distal side further than a distal surface 25 of the second tubular body 24. The step portion 26 can have a circular tubular shape. A proximal portion of the balloon 30 is fixed to an outer peripheral surface of the step portion 26. In addition, a connection section 54 between the electrode portion 40 and the conductor 50 may be disposed on the outer peripheral surface of the step portion 26. In addition, the conductors 50 can be equally disposed in a circumferential direction Z since the conductors 50 are disposed on an outer surface of the first tubular body 23 disposed inside the second tubular body 24.

A guide wire lumen 27 extending along an axial direction is formed inside the inner tube 22. A guide wire can be inserted into the guide wire lumen 27. An inflation lumen 28 is formed inside the outer tube 21 and outside the inner tube 22. An inflation fluid for inflating the balloon 30 can flow through the inflation lumen 28. The inflation fluid may be gas or a liquid. For example, the gas can be, for example, helium, CO², O², and nitrous oxide (i.e., laughing gas), a liquid such as a saline solution, a contrast agent, and a mixture of gases.

The inner tube 22 extends further to a distal side than a distal end of the first tube body 23. A distal portion of the balloon 30 can be fixed to an outer peripheral surface of the inner tube 22 on a distal side from the first tubular body 23. In the inner tube 22, a fixing portion 29 for fixing a distal portion of the electrode portion 40 is fixed to an outer peripheral surface on a distal side from a position where the balloon 30 is fixed.

An outer diameter of the shaft portion 20 is not particularly limited. However, it is preferable that the outer diameter of the shaft portion 20 is minimally invasive and is not excessively large to satisfy compatibility with a general sheath or a guiding catheter to be inserted. For example, the outer diameter of the shaft portion 20 is 4.0 mm or smaller, and is preferably 2.9 mm or smaller.

It is preferable that a material for forming the first tubular body 23, the second tubular body 24, and the inner tube 22 has flexibility to some degrees. In addition, it is preferable that the material for forming of the first tubular body 23, the second tubular body 24, and the inner tube 22 has an insulation property. Examples of the materials for forming the first tubular body 23, the second tubular body 24, and the inner tube 22 can include a polyolefin such as polyethylene, polypropylene, polybutene, ethylene-propylene copolymer, ethylene-vinyl acetate copolymer, ionomer, and a mixture of two or more types of polyolefin, and soft polyvinyl chloride resin, polyamide, polyamide elastomer, polyimide, polyester, polyester elastomer, polyurethane, fluororesin such as polytetrafluoroethylene, silicone rubber, and latex rubber.

In accordance with an exemplary embodiment, the balloon 30 is flexible and deformable. A shape of the balloon 30 is not particularly limited. However, for example, the shape of the balloon 30 may be a cylinder, an ellipsoid, or a trapezoid. The distal portion of the balloon 30 can be fixed to the outer peripheral surface of the distal portion of the inner tube 22. The proximal portion of the balloon 30 is fixed to the outer peripheral surface of the distal portion of the first tubular body 23. It is preferable that the balloon 30 is made from a relatively thin material having a thin film shape and flexibility. In addition, the balloon 30 needs to be strong enough to reliably spread the electrode portion 40. As a material for forming the balloon 30, the above-described materials for forming the shaft portion 20 can be used. Alternatively, for example, various elastomer materials such as hydrogenated styrene-based thermoplastic elastomer (SEBS) can be used for forming the balloon 30.

In accordance with an aspect, the conductor 50 has a linear shape. As illustrated in FIGS. 1 to 4, the conductor 50 has an embedded portion 52 embedded inside the outer tube 21, a protruding portion 51 protruding in a distal direction from the distal surface 25 of the outer tube 21, and a lead-out portion 53 which leads out (i.e., extends) from the outer tube 21 on the proximal side. The embedded portion 52 is interposed between the first tubular body 23 and the second tubular body 24. The embedded portion 52 is located outside an inner peripheral surface of the outer tube 21, and is located inside the outer peripheral surface of the outer tube 21. For example, a wire rod serving as the conductor 50 is wound around the outer peripheral surface of the first tubular body 23 and the second tubular body 24 is extruded outward. In this manner, the embedded portion 52 is embedded in the outer tube 21. It is preferable that the embedded portion 52 is covered by a material of the outer tube 21 formed of an insulation material without any gap. In this manner, it is possible to reliably suppress electrical short-circuit of the embedded portions 52 which may come into contact with each other. The number of the conductors 50 is equal to or more than the number of the electrode portions 40. In this manner, the conductor 50 can transmit an independent current to all of the electrode portions 40. In the present embodiment, the number of the conductors 50 is the same as the number of the electrode portions 40 (for example, ten (10). The plurality of conductors 50 are formed in a structure which is spirally wound multiple times. The plurality of conductors 50 are disposed away from each other at equal intervals. Therefore, the plurality of the conductors 50 can transmit the independent current without any electrical short-circuit. A separation distance between the conductors 50 is preferably set in accordance with a supplied electric signal so that the electrical short-circuit does not occur. For example, when an electric signal of 1,000 V is applied, the separation distance between the conductors 50 is preferably 10 μm or greater. In this manner, electrical safety of the medical device 10 can be improved.

When a radius (distance) from an axis X of the shaft portion 20 to a central axis Y of the conductor 50 is defined as R, the number of the conductors 50 is defined as N, and a tilting angle of the conductor 50 with respect to a cross section orthogonal to the axis X of the shaft portion 20 is defined as θ, a distance D between the central axes Y of the adjacent conductors 50 is 2 πR/N·sinθ.

In accordance with an embodiment, for example, the radius R can be 0.2 mm to 2.6 mm. For example, the number N can be 2 to 20. For example, the tilting angle θ can be 1 degrees to 45 degrees. For example, the distance D can be 0.02 mm to 8 mm.

In accordance with an exemplary embodiment, the protruding portion 51 can be substantially parallel to the axis X, and is connected to the proximal portion of the electrode portion 40. At least a portion of the protruding portion 51 is perpendicular to the circumferential direction Z of the shaft portion 20. That is, at least a portion of the protruding portion 51 is parallel to the axis X of the shaft portion, when a development view of the shaft portion 20 developed in the circumferential direction Z is viewed from the outside in the radial direction of the shaft portion 20 (refer to FIG. 4). Therefore, when the protruding portion 51 protrudes in the distal direction from the embedded portion 52 spirally wound inside the outer tube 21 through the distal surface 25, the angle is changed, and the protruding portion is substantially parallel to the axis X. The protruding portion 51 may not be parallel to the axis X. For example, the protruding portion 51 may be tilted in the distal direction to be closer to the axis X of the shaft portion 20. Even in this case, the protruding portion 51 can be perpendicular to the circumferential direction Z. The distal end of the protruding portion 51 is located on the proximal side from the distal end of the step portion 26. Therefore, the proximal portion of the protruding portion 51 and the electrode portion 40 can be stably disposed on an outer surface of the step portion 26.

The lead-out portion 53 is drawn out (i.e., extends) in a proximal direction from the proximal portion of the second tubular body 24, and is connected to a power source unit 12 provided outside the shaft portion 20. The power source unit 12 can supply electricity to the electrode portion 40.

A material for forming the conductor 50 is preferably highly conductive. For example, copper, gold, platinum, silver, aluminum, alloy, or carbon fiber may be used for the material for forming the conductor 50. As the conductor 50, a known lead wire can be used.

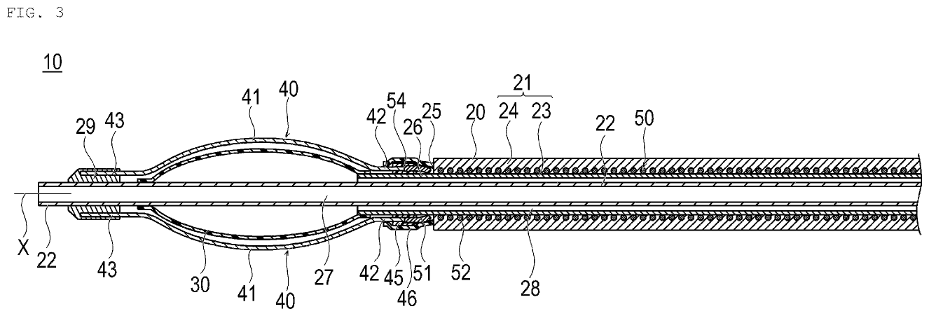

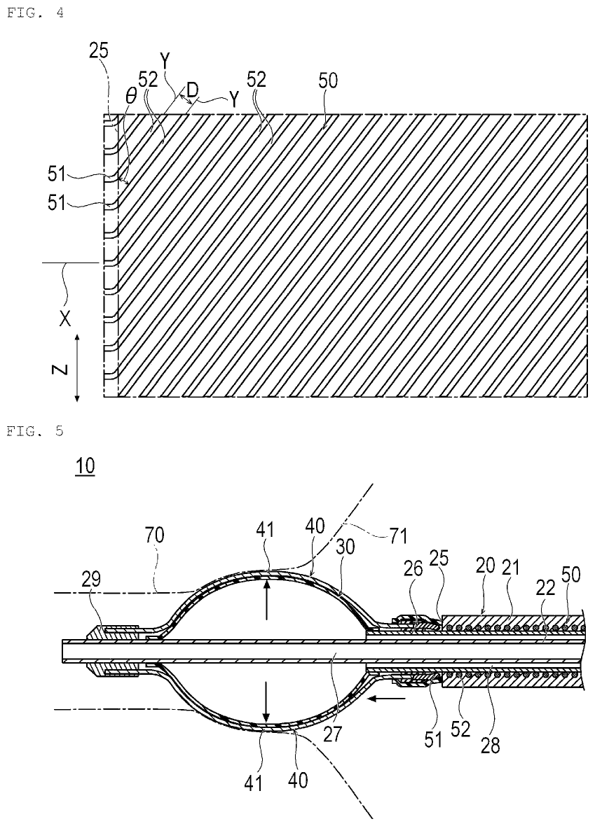

As illustrated in FIGS. 2A, 2B, and 3, each of the electrode portions 40 is located on an outer peripheral side of the balloon 30, and is not fixed to the balloon 30. The electrode portion 40 may be fixed to the balloon 30. The electrode portion 40 is formed of a wire rod which is conductive and flexible. The plurality of electrode portions 40 are disposed to be aligned in the circumferential direction Z of the balloon 30 on the outer peripheral side of the balloon 30. Each of the electrode portions 40 extends in the axial direction of the shaft portion 20. The electrode portion 40 includes a curved portion 41 that can be curved in the radial direction of the shaft portion 20. The electrode portion 40 has an electrode proximal portion 42 on the proximal side from the balloon 30 and an electrode distal portion 43 on the distal side from the balloon 30.

In accordance with an exemplary embodiment, a proximal end of the electrode proximal portion 42 is in contact with and joined to a distal end of the protruding portion 51 of the conductor 50. In this manner, the electrode portion 40 is electrically connected to the conductor 50. The connection section 54 between the electrode proximal portion 42 and the protruding portion 51 which are provided in each of the electrode portions 40 is covered by an insulation tube 45 formed of an insulation material. A joining method is not limited as long as the method enables electrical conduction. For example, soldering, laser fusion, welding using various metal braces, bonding using a conductive adhesive, or mechanical interlocking using a chuck may be used. The plurality of connection sections 54, the insulation tube 45, and the protruding portion 51 which are aligned on the outer peripheral surface of the step portion 26 are collectively covered by a single protective tube 46 formed of an insulation material. Therefore, the connection section 54 and the protruding portion 51 are covered by the insulation tube 45 and/or the protective tube 46 on the distal side from the distal surface 25, and are not exposed outward. In this manner, electrical safety of the medical device 10 can be improved. As a material for forming the insulation tube 45 and the protective tube 46, the above-described materials for forming the shaft portion 20 can be used.

The electrode distal portion 43 is fixed to the fixing portion 29 provided on the distal side from the balloon 30 of the inner tube 22. In accordance with an exemplary embodiment, a cross-sectional shape orthogonal to the axial direction of each of the electrode portions 40 can be rectangular. That is, the cross-sectional shape orthogonal to the axial direction of the electrode portion 40 is a shape formed so that in at least a portion of the electrode portion 40, the length along the circumferential direction Z of the balloon 30 is longer than the length along the radial direction of the balloon 30. Therefore, a long side of the cross section extends along the circumferential direction Z of the balloon 30. In this manner, the plurality of electrode portions 40 aligned in the circumferential direction Z of the balloon 30 are likely to be bent in the radial direction of the balloon 30, and are less likely to deform in a direction in which the electrode portions 40 are closer to each other. Therefore, electrical short-circuit or entanglement between the electrode portions 40 can be suppressed. A cross-sectional shape of the electrode portion 40 is not limited to a rectangle, and for example, may be a circle, a semicircle, an ellipse, or a square.

As a material for forming the electrode portion 40, for example, superelastic metal represented by a Ni—Ti alloy can be preferably used. However, the electrode portion 40 may be formed of a conductive material other than the above-described material. For example, the electrode portion 40 may be conductive rubber. Furthermore, the electrode portion 40 may be formed of a flexible printed circuit board (FPC).

In the electrode portion 40, the outer surface other than the curved portion 41 can be coated with an insulation material. The insulation material is not electrically conductive. The insulation material may be provided on a side where the outer surface of the electrode portion 40 is not in contact with the biological tissue, that is, a side that faces the balloon 30.

In the present exemplary embodiment, ten (10) electrode portions 40 are equally provided in the circumferential direction Z. However, the number of the electrode portions 40 may be larger or smaller than 10. An electric signal is applied between the adjacent electrode portions 40. However, an electrode (alternatively, a counter electrode) may be disposed outside a body, and the electric signal may be applied between the electrode (alternatively, the counter electrode) outside the body and the electrode portion 40.

As illustrated in FIG. 1, the proximal portion of the inner tube 22 is interlocked with the hub 60. The first tubular body 23 located outside the inner tube 22 and movable in the axial direction with respect to the inner tube 22 is interlocked with the hub 60 to be slidable. The hub 60 has a first port 61 having an opening communicating with the guide wire lumen 27 and a second port 62 having an opening communicating with the inflation lumen 28.

Next, a treatment method using the medical device 10 will be described. First, an introducer percutaneously punctures the blood vessel. Next, after a guide wire is inserted into a guiding catheter, the guiding catheter is inserted into the introducer. Next, the guide wire is protruded to the distal side, and thereafter, a distal portion of the guiding catheter is inserted into the blood vessel through a distal portion opening of the introducer. Thereafter, while the guide wire is moved ahead, the guiding catheter is gradually pushed to a target site. An operator forms a through-hole in an atrial septum by penetrating a predetermined puncture device from a right atrium side toward a left atrium side. For example, as the puncture device, a device such as a wire having a sharp distal end can be used. The puncture device can be delivered via the guiding catheter. In addition, for example, the puncture device can be delivered into the atrial septum instead of the guide wire after the guide wire is removed from the guiding catheter. A specific structure of the puncture device used for penetrating the atrial septum, and a specific procedure for forming the through-hole is not particularly limited. After the through-hole is formed, the operator uses a dilator to widen the through-hole. Next, the operator causes the guiding catheter to pass through the through-hole, and uses the guide wire to push the guiding catheter forward to the target site (for example, vicinity of the pulmonary vein).

Next, an end of the guide wire is inserted into a distal opening portion of the guide wire lumen 27 of the shaft portion 20, and the guide wire is pulled out from the first port 61 of the hub 60. Next, the medical device 10 is inserted from the distal portion into the guiding catheter inserted into the blood vessel, and the medical device is pushed forward along the guide wire. At this time, a ring catheter provided with an electrode may be used instead of the guide wire.

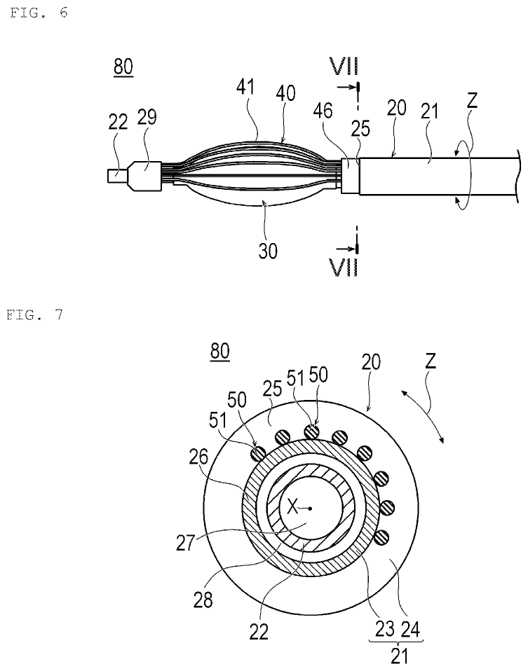

As illustrated in FIG. 5, after the electrode portion 40 is inserted into an entrance of a pulmonary vein 70 which is a target position, the inflation fluid is supplied into the balloon 30 via the second port 62 and the inflation lumen 28. In this manner, the balloon 30 is inflated, and the electrode portion 40 pushed by the balloon 30 is expanded in the radial direction. At this time, the outer tube 21 moves to the distal side with respect to the inner tube 22, and the proximal portion of the electrode portion 40 moves to the distal side. In this manner, the electrode portion 40 can deform while following the inflation of the balloon 30. Therefore, the curved portion 41 located in a central portion of the electrode portion 40 is pushed against a biological wall 71 by the balloon 30. The electrode portion 40 can deform to fit a shape of the biological tissue. Therefore, the electrode portion 40 can be brought into contact with the biological tissue, and an electric signal is likely to be applied. In this state, the electric signal is applied from the power source unit 12 to the electrode portion 40 via the conductor 50.

A pulsed electric signal is first applied from the power source unit 12 to a pair of electrode portions 40 adjacent to each other in the circumferential direction Z. In this manner, a current flows between the pair of electrode portions 40 adjacent to each other in the circumferential direction Z. Next, the pulsed electric signal is applied to the other pair of electrode portions 40 adjacent to each other in the circumferential direction Z. The electric signals are sequentially applied to all pairs of the electrode portions 40 adjacent to each other in the circumferential direction Z. An example of the applied electric signal will be described below. Electric field intensity applied by the power source unit 12 can be, for example, 1,500 V/cm, and a pulse width of the electric signal can be 100 μsec. The electric signals are repeatedly applied to all pairs of the electrode portions 40 adjacent to each other in the circumferential direction Z, for example, 60 times to 180 times in a cycle of once every 2 seconds, depending on a refractory period of a ventricular muscle. In this manner, cells in the entrance of the pulmonary vein are necrotized over the entire periphery. The electric signal may be applied between the plurality of electrode portions 40 that are not adjacent to each other, or the electric signal may be applied from the electrode portion 40 to a counter electrode attached to a body surface.

When the electric signal is completely applied, the balloon 30 is deflated. In this manner, the electrode portion 40 is contracted in the radial direction due to a self-restoring force. At this time, the outer tube 21 moves to the proximal side with respect to the inner tube 22, and the proximal portion of the electrode portion 40 moves to the proximal side. In this manner, the electrode portion 40 can deform while following the deflation of the balloon 30. Thereafter, all instruments inserted into the blood vessels are removed to complete the procedure. When the electrode portion 40 is formed of a material other than the superelastic alloy such as the Ni—Ti alloy, it is preferable to perform an operation for contracting the electrode portion 40 in the radial direction by pushing the inner tube 22 to the distal side (or by pulling the outer tube 21 to the proximal side).

As described above, the medical device 10 according to the first exemplary embodiment includes the elongated shaft portion 20, the plurality of electrically independent electrode portions 40 disposed on the distal portion of the shaft portion 20, extending along the length direction of the shaft portion 20, and deformable in the radial direction of the shaft portion 20, and the plurality of electrically independent conductors 50 including the embedded portion 52 embedded in the shaft portion 20, and allowing the current to flow to the electrode portions 40. In accordance with an exemplary embodiment, at least one of the conductors 50 has the protruding portion 51 protruding from the distal surface 25 of the shaft portion 20, and connected to the electrode portion 40.

In the medical device 10 configured as described above, the embedded portion 52 on the proximal side of the plurality of conductors 50 can be embedded in the shaft portion 20. The protruding portion 51 on the distal side connected to the electrode portion 40 protrudes from the distal surface 25 of the shaft portion 20. Therefore, the electrode portion 40 can be located inside the outer diameter of the shaft portion 20. Accordingly, the electrode portion 40 does not protrude outward in the radial direction, and can have a reduced diameter. Therefore, the medical device 10 can be inserted into a relatively narrow biological lumen, and a wide range can be effectively ablated by the electrode portion 40 curved in the radial direction. The electrode portion 40 may be located at a position the same as that of the outer peripheral surface of the shaft portion 20. Furthermore, the conductor 50 is embedded in the shaft portion 20. Accordingly, bending rigidity of the shaft portion 20 can be increased, and kink resistance can be improved. For example, when the electrode portion 40 is brought into contact with the vicinity of the entrance of the pulmonary vein 70, the shaft portion 20 cannot move in the radial direction in a puncture site of the atrial septum (for example, an oval fossa). Accordingly, the shaft portion 20 is curved. At this time, since the bending rigidity of the shaft portion 20 is increased, kink of the shaft portion 20 can be suppressed.

In addition, each of the plurality of conductors 50 has the protruding portion 51, and each of the protruding portions 51 is connected to the different electrode portion 40. In this manner, the independent electric signal can be applied to each of the plurality of electrode portions 40.

In addition, the protruding portion 51 is connected to the electrode portion 40 located on the distal side of the protruding portion 51 in the axial direction. In this manner, the electrode portion 40 does not protrude outward in the radial direction, and the medical device 10 can rather easily have the reduced diameter.

In addition, at least a portion of the protruding portion 51 is perpendicular to the circumferential direction Z of the shaft portion 20. In this manner, at least one of the electrode portion 40 and the protruding portion 51 can be accurately disposed at a suitable position in the circumferential direction Z of the shaft portion 20.

In addition, at least a portion of the protruding portion 51 is parallel to the axis X of the shaft portion 20, and at least a portion of the embedded portion 52 has a spiral shape wound around the axis X of the shaft portion 20. In this manner, the bending rigidity of the shaft portion 20 is not biased by a bending direction, and the shaft portion 20 having uniform quality can be formed.

In addition, the plurality of electrode portions 40 are equally disposed in the circumferential direction Z of the shaft portion 20. In this manner, the electrode portion 40 can evenly ablate the target site.

In addition, when the radius from the axis X of the shaft portion 20 to the conductor 50 is defined as R, the number of the conductors 50 is defined as N, and the tilting angle of the conductor 50 with respect to the cross section orthogonal to the axis X of the shaft portion 20 is defined as θ, the distance D between central axes Y of the conductors 50 adjacent to each other is 2 πR/N·sinθ. In this manner, the conductors 50 can be equally disposed in the circumferential direction Z of the shaft portion 20. Therefore, when the electrode portions 40 are equally disposed in the circumferential direction Z, the electrode portions 40 are rather easily disposed so that the position of the conductor 50 is aligned with the electrode portion 40.

In addition, the shaft portion 20 has the step portion 26 protruding to the distal side of the distal surface 25 from a position inside the distal surface 25 in the radial direction. In this manner, the protruding portion 51 of the conductor 50 can be supported by the step portion 26, and disconnection of the conductor 50 can be suppressed. In the present embodiment, the outer tube 21 of the shaft portion 20 has the first tubular body 23 including the step portion 26, and the second tubular body 24 covering the outer peripheral surface of the first tubular body 23 and fixed to the first tubular body 23. Accordingly, the step portion 26 from a position inside the distal surface 25 in the radial direction can be rather easily formed.

Also, the medical device 10 has the expansion body (for example, the balloon 30) located between the shaft portion 20 and the electrode portion 40, and inflatable outward in the radial direction of the shaft portion 20. In this manner, the medical device 10 can bring the electrode portion 40 into relatively close contact with the biological tissue by inflating the expansion body.

### Second Embodiment

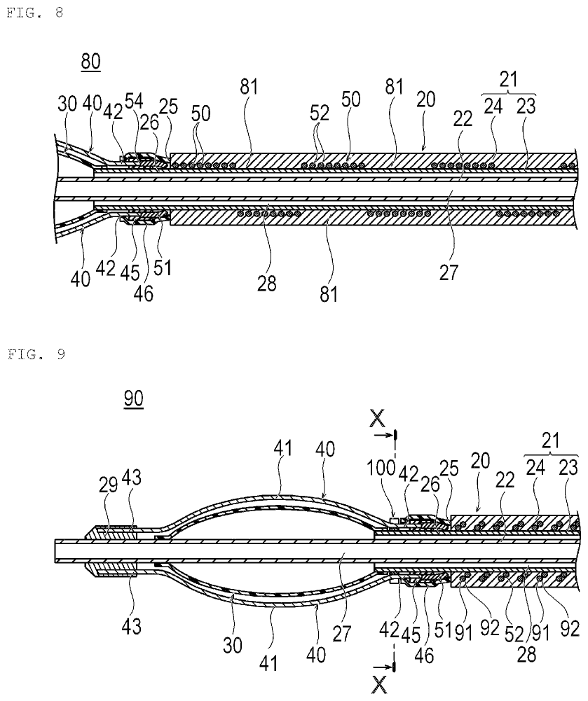

A medical device 80 according to a second exemplary embodiment is different from that according to the first exemplary embodiment only in the following point. As illustrated in FIGS. 6 to 8, the electrode portion 40 and the conductor 50 are provided to be biased in the circumferential direction Z of the shaft portion 20.

The plurality of the conductors 50 have the protruding portion 51 protruding from the distal surface 25 of the shaft portion 20 in the distal direction at a position biased in the circumferential direction Z of the shaft portion 20. Therefore, the plurality of conductors 50 inside the shaft portion 20 collectively form one group, and are disposed in a spiral shape while a gap 81 is interposed between the plurality of conductors 50 in a collective state. The protruding portion 51 and the electrode proximal portion 42 of the plurality of electrode portions 40 are located on the distal side of the plurality of the protruding portions 51. Therefore, it is rather easy to electrically connect the electrode proximal portion 42 to the protruding portion 51.

As described above, in the medical device 80 according to the second exemplary embodiment, the plurality of electrode portions 40 are disposed to be biased to a portion of the shaft portion 20 in the circumferential direction Z. In this manner, in the medical device 80, only a specific site in the circumferential direction Z can be intentionally ablated by the electrode portion 40. Therefore, in the medical device 80, it is possible to rather easily adjust the electrode portion 40 so that only the specific site is ablated and other sites are not ablated. A biasing method of the protruding portion 51 is not particularly limited. Therefore, the protruding portion 51 and the electrode portion 40 may be provided to be biased at a plurality of locations in the circumferential direction Z of the shaft portion 20. In addition, the distance between the adjacent protruding portions 51 and the distance between the adjacent electrode portions 40 may not be uniform.

### Third Embodiment

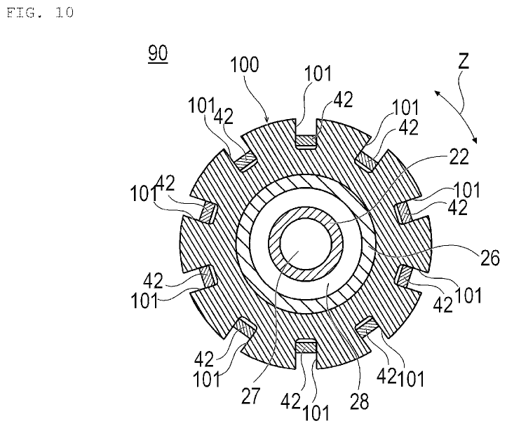

A medical device 90 according to a third exemplary embodiment is different from that according to the first embodiment only in that the conductor 50 is braided, and has a support body 100 as illustrated in FIGS. 9 and 10.

In accordance with an exemplary embodiment, a plurality of braided wire rods 91 are disposed inside the shaft portion 20. A portion of the wire rods 91 is used as the conductor 50. All of the wire rods 91 may be used as the conductors 50. The wire rod 91 used as the conductor 50 is formed of a conductive material, and a surface of the wire rod 91 is coated with the insulation layer 92. The wire rod 91 which is not used as the conductor 50 may be formed of the conductive material, or may not be formed of the conductive material. When the wire rod 91 which is not used as the conductor 50 is not formed of the conductive material, the electrical short-circuit with the wire rod 91 which is used as the conductor 50 can be suppressed. In the wire rod 91 which is not used as the conductor 50, the surface may be coated with or may not be coated with the insulation layer 92. The support body 100 that supports the electrode proximal portion 42 of the plurality of electrode portions 40 is fixed to an outer surface of the step portion 26 of the shaft portion 20.

The support body 100 may have a tubular shape, and a plurality of housing portions 101 that house the electrode proximal portions 42 of the electrode portions 40 are formed on the outer peripheral surface. The support body 100 is located away from the distal surface 25 in the distal direction. The support body 100 may not be located away from the distal surface 25 in the distal direction. In accordance with an embodiment, the housing portion 101 is a groove extending in the axial direction of the shaft portion 20. The plurality of housing portions 101 can be formed to be equally aligned in the circumferential direction Z of the support body 100. Each of the housing portions 101 houses one of the electrode proximal portions 42. Each of the electrode proximal portions 42 may be fixed to the support body 100 by using an adhesive in a state of being housed in the support body 100. The support body 100 may house the protruding portion 51 of the conductor 50 instead of the electrode proximal portion 42. Alternatively, the support body 100 may house both the electrode proximal portion 42 and the protruding portion 51. In addition, the support body 100 may be provided on the outer peripheral surface of the inner tube 22 located on the distal side from the balloon 30 to house the electrode distal portion 43 located on the distal side of the electrode portion 40.

As described above, the medical device 90 according to the third exemplary embodiment further has the annular support body 100 disposed on the outer peripheral surface of the shaft portion 20, and the support body 100 has the housing portion 101 that houses at least one of the electrode portion 40 and the protruding portion 51. In this manner, at least one of the electrode portion 40 and the protruding portion 51 can be accurately disposed at a suitable position in the circumferential direction Z of the shaft portion 20.

In addition, the support body 100 is located away from the distal surface 25 in the distal direction. In this manner, the protruding portion 51 protruding from the distal surface 25 has a suitable posture in the gap between the support body 100 and the distal surface 25, and is disposed at a suitable position of the support body 100.

In addition, the conductor 50 is at least a portion of the plurality of wire rods 91 braided in a spiral shape, and the surface is covered with the insulation layer 92. In this manner, the conductor 50 can use the braided wire rod 91, and even when the conductors 50 come into contact with each other due to the braiding, the insulation layer 92 can help suppress the electrical short-circuit.

The present disclosure is not limited to the above-described embodiments, and various modifications can be made by those skilled in the art within the technical idea of the present disclosure. For example, a configuration has been described in which the medical device 10 according to the above-described embodiments is used for treating the pulmonary vein. However, the medical device 10 may be used for treating other sites, for example, such as a renal artery, an ascending vena cava, and a ventricle.

In addition, the conductor 50 which transmits the current to the electrode portion 40 may not have a spiral shape, or may not be a portion of the braided wire rod 91. For example, the conductor 50 may be the wire rod linearly extending along the axis X of the shaft portion 20. In addition, the conductor 50 may be the wire rod that is curved or bent.

In addition, the expansion body of the medical device may not be the balloon 30. In addition, the medical device may not have the expansion body that presses the electrode portion 40 outward in the radial direction. For example, the medical device can expand the electrode portion 40 in the radial direction of the shaft portion 20 by moving the outer tube 21 with respect to the inner tube 22 in the distal direction without being provided with the balloon 30. In addition, when the electrode portion 40 or a portion of the conductor 50 is provided with an extendable portion, the outer tube 21 and the inner tube 22 may not be relatively movable in the axial direction. In this case, even when the balloon 30 is inflated and the electrode portion 40 is curved outward in the radial direction, the extendable portion extends. Accordingly, the outer tube 21 and the inner tube 22 do not need to relatively move. Alternatively, the fixing portion 29 may be slidable in the axial direction with respect to the outer peripheral surface of the inner tube 22. In addition, the conductor may be embedded in the inner tube 22 instead of the outer tube 21. In this case, the conductor is electrically connected to the distal portion of the electrode portion 40.

The detailed description above describes embodiments of a medical device inserted into a living body to perform ablation treatment on a biological tissue. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
an elongated shaft;
a plurality of electrically independent electrodes disposed on a distal side of the elongated shaft, the plurality of electrically independent electrodes extending along an axial direction of the elongated shaft and deformable in a radial direction of the elongated shaft;
a plurality of electrically independent conductors, each of the plurality of electrically independent conductors including an embedded portion embedded in the elongated shaft, and the plurality of electrically independent conductors configured to allow a current to flow to the plurality of electrically independent electrodes; and
wherein at least one of the plurality of electrically independent conductors has a protruding portion protruding from a distal surface of the elongated shaft portion, and wherein the at least one of the plurality of electrical independent conductors is connected to one of the plurality of electrically independent electrodes.

2. The medical device according to claim 1, wherein each of the plurality of electrically independent conductors has the protruding portion, and each of the protruding portions is connected to a different one of the plurality of electrically independent electrode portions.

3. The medical device according to claim 1, wherein at least a portion of the protruding portion is perpendicular to a circumferential direction of the elongated shaft portion.

4. The medical device according to claim 1, wherein at least a portion of the protruding portion is parallel to an axis of the elongated shaft portion, and at least a portion of the embedded portion has a spiral shape wound around the axis of the elongated shaft portion.

5. The medical device according to claim 1, wherein the plurality of electrically independent electrodes are equally disposed in a circumferential direction of the elongated shaft.

6. The medical device according to claim 1, wherein when a radius from an axis of the elongated shaft to each of the plurality of electrically independent conductors is defined as R, a number of the plurality of electrically independent conductors is defined as N, and a tilting angle of the each of the plurality of electrically independent conductors with respect to a cross section orthogonal to the axis of the elongated shaft is defined as θ, a distance between central axes of the plurality of electrically independent conductors adjacent to each other is 2 πR/N·sinθ.

7. The medical device according to claim 1, wherein the plurality of electrically independent electrodes is disposed to be biased to a portion of the elongated shaft in a circumferential direction.

8. The medical device according to claim 1, further comprising:
an annular support body disposed on an outer peripheral surface of the elongated shaft, and wherein the support body has a housing that houses at least one of the plurality of electrically independent electrodes and the protruding portion.

9. The medical device according to claim 1, wherein the elongated shaft has a step portion protruding from a position inside the distal surface in the radial direction to a distal side of the distal surface.

10. The medical device according to claim 1, wherein the plurality of electrically independent conductors is at least a portion of a plurality of spirally braided wire rods, and a surface of the plurality of spirally braided wire rods is coated with an insulation material.

11. The medical device according to claim 1, further comprising:
an expansion body located between the elongated shaft and the plurality of electrically independent electrodes, and inflatable outward in the radial direction of the elongated shaft.

12. A medical device comprising:
an elongated shaft that includes an outer tubular body and an inner tubular body, the inner tubular body extending inside of the outer tubular body and along the outer tubular body;
a plurality of electrically independent electrodes disposed distal of the elongated shaft portion, the plurality of electrically independent electrodes extending along an axial direction of the shaft portion and deformable in a radial direction of the elongated shaft;
a plurality of electrically independent conductors including a portion extending along the shaft portion and disposed between an outer surface of the inner tube body and an inner surface of the outer tube body, the portion of the plurality of electrically independent conductors extending along the elongated shaft being configured to allow a current to flow to at least one of the plurality of electrically independent electrodes;
the inner tubular body including an extending portion that extends distal of the distal end of the outer tubular body; and
wherein at least one of the plurality of electrically independent conductors includes a protruding portion protruding from the distal end of the outer tubular body, and the protruding portion is connected to at least one of the plurality of electrically independent electrodes on the extending portion of the inner tubular body.

13. The medical device according to claim 12, wherein each of the plurality of electrically independent conductors has the protruding portion, and each of the protruding portions is connected to a different one of the plurality of electrically independent electrodes.

14. The medical device according to claim 12, wherein at least a portion of the protruding portion is perpendicular to a circumferential direction of the elongated shaft portion.

15. The medical device according to claim 12, wherein at least a portion of the protruding portion is parallel to an axis of the elongated shaft portion, and at least a portion of the portion has a spiral shape wound around the axis of the elongated shaft portion.

16. The medical device according to claim 12, wherein the plurality of electrically independent electrodes are equally disposed in a circumferential direction of the elongated shaft.

17. The medical device according to claim 12, wherein when a radius from an axis of the elongated shaft to each of the plurality of electrically independent conductors is defined as R, a number of the plurality of electrically independent conductors is defined as N, and a tilting angle of the each of the plurality of electrically independent conductors with respect to a cross section orthogonal to the axis of the elongated shaft is defined as θ, a distance between central axes of the plurality of electrically independent conductors adjacent to each other is 2 πR/N·sinθ.

18. The medical device according to claim 12, further comprising:
an annular support body disposed on an outer peripheral surface of the elongated shaft, and wherein the support body has a housing that houses at least one of the plurality of electrically independent electrodes and the protruding portion.

19. A medical device comprising:
an elongated shaft that includes a first tubular body;
a plurality of electrodes disposed distal of the shaft portion, the plurality of electrodes extending along a length direction of the elongated shaft and deformable in a radial direction of the shaft portion;
a plurality of conductors extending on an outer surface of the first tubular body along the shaft portion and include connection sections at distal end portions of the conductors, the connection sections electrically connected to proximal end sections of the plurality of electrodes;
an annular support body disposed on an outer peripheral surface of the first tubular body;
an insulation tube formed of an insulation material and disposed on the annular support body;
the support body including a plurality of housing portions aligned in a circumferential direction of the support body, the housing portions housing the proximal end sections of the plurality of electrodes; and
wherein the proximal end sections of the plurality of electrodes and the connection sections of the plurality of conductors are disposed on the first tubular body and covered together with the support body by the insulation tube.

20. The medical device according to claim 19, wherein each of the plurality of conductors has the protruding portion, and each of the protruding portions is connected to a different one of the plurality of electrodes.
